(19) United States
(12) Patent Application Publication
(10) Pub. No.: US 2025/0325236 A1
(43) Pub. Date: Oct. 23, 2025

(54) Panatomic Imaging Derived 4D Hemodynamics Using Deep Learning

(71) Applicant: Northwestern University, Evanston, IL (US)

(72) Inventors: Bradley Allen, Evanston, IL (US); Michael Markl, Chicago, IL (US); Ulas Bagci, Chicago, IL (US)

(21) Appl. No.: 18/865,562

(22) PCT Filed: May 5, 2023
(86) PCT No.: PCT/US2023/066652

§ 371 Date: Nov. 13, 2024

Related U.S. Application Data
(60) Provisional application No. 63/346,956, filed on May 30, 2022.

(51) Int. Cl. A61B 5/00; A61B 5/02; A61B 5/055; G06T 7/00; G06T 7/215; G06V 10/26; G06V 10/764; G06V 10/82; G06V 10/94

(52) U.S. Cl. CPC A61B 5/7267; A61B 5/02007; A61B 5/055; G06T 7/0014; G06T 7/215; G06V 10/764; G06V 10/82; G06V 10/95; A61B 2576/02; G06T 2200/24; G06T 2207/10076; G06T 2207/10088; G06T 2207/20081; G06T 2207/20084; G06T 2207/30104; G06V 10/26; G06V 2201/03

(57) ABSTRACT

A method for non-invasive assessment of vascular 4D hemodynamics includes receiving standard anatomic imaging data at a local network or cloud-based analysis platform and identifying a vessel of interest from the received anatomic imaging data. The method also includes deriving hemodynamic features from the vessel of interest from the received anatomic imaging data using deep learning by inputting the received anatomic imaging data into a deep learning network. The method further includes calculating 4D hemodynamic parameters and generating output data based on the hemodynamic features derived from the vessel of interest.

### CROSS REFERENCE TO RELATED APPLICATIONS

The present application claims the benefit of priority under 35 U.S.C. § 119 from U.S. Provisional Patent Application Ser. No. 63/346,956, entitled “Vascular Imaging Derived 4D Hemodynamics Using Deep Learning,” filed on May 30, 2022, all of which is incorporated herein by reference in its entirety for all purposes.

### TECHNICAL FIELD

The present disclosure generally relates to medical imaging, and more specifically relates to 4D hemodynamics derived directly form anatomic imaging data using artificial intelligence concepts such as deep learning.

### BACKGROUND

Vascular hemodynamic characterization and associated risk-stratification is increasingly recognized as an important tool in treating numerous vascular diseases. However, the reliable measurement and calculation of hemodynamic parameters is often challenging and frequently not performed due to limited availability of appropriate equipment and software, increased time and cost to acquire additional dedicated imaging, cumbersome and time-consuming data analysis, and lack of local technical expertise at most medical centers.

The description provided in the background section should not be assumed to be prior art merely because it is mentioned in or associated with the background section. The background section may include information that describes one or more aspects of the subject technology.

### SUMMARY

An exemplary method for non-invasive assessment of vascular 4D hemodynamics includes receiving standard anatomic imaging data at a local network or cloud-based analysis platform and identifying a vessel of interest from the received anatomic imaging data. The method also includes deriving hemodynamic features from the vessel of interest from the received anatomic imaging data using deep learning by inputting the received anatomic imaging data into a deep learning network. The method further includes calculating 4D hemodynamic parameters and generating output data based on the hemodynamic features derived from the vessel of interest.

Identifying the vessel of interest may include pre-processing the anatomic imaging data that is received. Identifying the vessel of interest may include performing 3D segmentation of the vessel of interest.

The method may further include passing the received anatomic images to a deep learning network for performing 4D hemodynamic quantification and pre-processing of anatomic imaging data of the vessel of interest on the deep learning network.

The method may further include training the deep learning network using expert labeled datasets of previously obtained vascular imaging data.

The method may further include deriving spatially and temporally resolved 3D blood flow velocities in the vessel of interest from the received anatomic imaging data using deep learning by inputting the received anatomic imaging data into a deep learning network.

Calculating 4D hemodynamic parameters may be performed on a deep learning network from anatomic imaging data.

The deep learning network may be trained using expert-analyzed 4D flow MRI data as ground truth data.

The method may further include displaying the output data that is generated on a device selected from the group consisting of an image viewer, a picture archiving and communication system. and a graphical user interface.

The graphical user interface may be configured to facilitate at least one of quantitative interrogation, cine review, and multiplanar reformation.

An exemplary system for non-invasive assessment of vascular 4D hemodynamics includes at least one device including a hardware computing processor, the system being configured to perform operations of a method for non-invasive assessment of vascular 4D hemodynamics as described herein. The system may include a non-transitory memory having stored thereon computing instructions, executable by the hardware computing processor, to perform operations of a method for non-invasive assessment of vascular 4D hemodynamics as described herein.

An exemplary system for non-invasive assessment of vascular 4D hemodynamics includes at least one device including a hardware circuit operable to perform a function, the system being configured to perform operations of a method for non-invasive assessment of vascular 4D hemodynamics as described herein.

### BRIEF DESCRIPTION OF DRAWINGS

The disclosure is better understood with reference to the following drawings and description. The elements in the figures are not necessarily to scale, emphasis instead being placed upon illustrating the principles of the disclosure. Moreover, in the figures, like-referenced numerals may designate to corresponding parts throughout the different views.

FIG. 1 illustrates emergence of a future paradigm in vascular imaging and hemodynamic assessment due to introducing one or more aspects of technology described herein for anatomic imaging derived 4D hemodynamics with deep learning to a current paradigm in vascular imaging and hemodynamic assessment for a chosen example case of thoracic aorta assessment with 4D flow MRI, according to one or more examples.

FIG. 2A illustrates an outline of an exemplary deep learning framework based on a CycleGAN neural network that may be employed to derive 4D hemodynamics from anatomic 3D segmentations of an aorta.

FIG. 2B illustrates exemplary hemodynamic maps of peak velocities of the 4D flow (4D flow MRI) and AI-Derived systolic velocities.

FIG. 2C illustrates an exemplary generator convolutional neural network (CNN) architecture.

FIG. 2D illustrates an exemplary discriminator convolutional neural network (CNN) architecture.

FIG. 3 illustrates an exemplary workflow for implementation of the subject technology for a chosen example case of thoracic aorta assessment.

FIGS. 4A and 4B illustrate exemplary results from a validation study based on a total of N=1765 patients (n=1242 bicuspid aortic valve [BAV] patients; n=523 trileaflet aortic valve [TAV] patients).

FIGS. 5A and 5B illustrate exemplary results of a validation study based on a total of N=1765 patients (n=1242 BAV patients; n=523 TAV patients).

FIG. 6 shows a table that illustrates exemplary results from a validation study based on a total of N=1765 patients (n=1242 BAV patients; n=523 TAV patients).

FIGS. 7A and 7B illustrate results from a validation study based on a total of N=720 patients (477 BAV patients; 243 TAV patients).

In one or more implementations, not all of the depicted components in each figure may be required, and one or more implementations may include additional components not shown in a figure, including alternative artificial intelligence concepts and/or deep learning network architecture. Variations in the arrangement and type of the components may be made without departing from the scope of the subject disclosure. Additional components, different components, or fewer components may be utilized within the scope of the subject disclosure.

### DETAILED DESCRIPTION

The detailed description set forth below is intended as a description of various implementations and is not intended to represent the only implementations in which the subject technology may be practiced. As those skilled in the art would realize, the described implementations may be modified in various different ways, all without departing from the scope of the present disclosure. Accordingly, the drawings and description are to be regarded as illustrative in nature and not restrictive.

Vascular disease is highly prevalent and associated with high morbidity and mortality. Examples include, but are not limited to, aneurysm, dissection, stenosis, and thoracic aorta disease. Vascular disease may occur in the arterial and/or venous vasculature throughout the body. Bicuspid aortic valve (BAV), for example, is associated with ascending aorta aneurysm and aortic coarctation, and is present in about 2% of the population. The risk of aortic aneurysm is up to 8-fold higher in BAV relative to the general population. Surgery for BAV is usually performed in patients with aortic size>5.5 cm or growth rates>3 mm/year. Similar aortic diameter-based risk-stratification metrics may be used in other aortopathies, such as type B aortic dissection (TBAD). The measurements may be performed on aorta images acquired with CT angiography (CTA) or MR angiography (MRA), for example. The imaging and measurement studies may be performed every 6-12 months in most patients having these aortopathies.

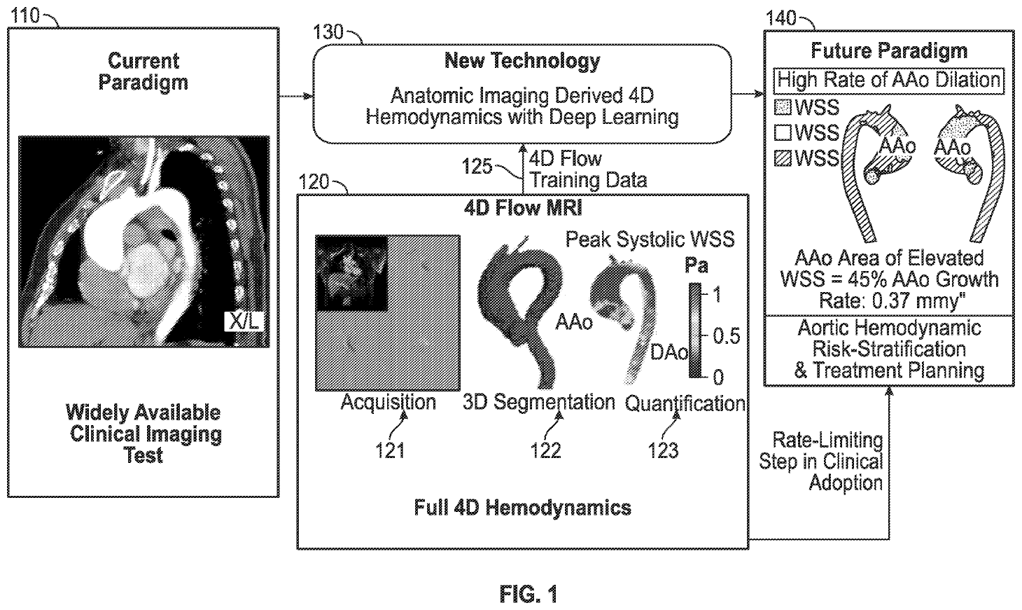

Medical imaging is frequently utilized for diagnosis, risk-stratification, and monitoring of vascular diseases. Most imaging techniques, including but not limited to computed tomography angiography (CTA) and magnetic resonance angiography (MRA), are used to evaluate anatomic and morphologic data (e.g., diameter of vessel). In general, MRA is an excellent imaging option for long-term patient follow-up due to its lack of ionizing radiation, better tissue characterization than CTA, and usefulness of a non-contrast imaging option for patients with poor kidney function. However, CTA is much more widely available compared to MRA, and also requires less technical and professional expertise for image acquisition and interpretation. CTA may be more commonly used by radiologists, surgeons, and other clinicians in the evaluation of acute and chronic aortic disease for both risk-stratification and surgery planning, as illustrated with reference to the traditional paradigm in FIG. 1.

FIG. 1 illustrates emergence of a future paradigm 140 in vascular imaging and hemodynamic assessment due to introducing one or more aspects of new technology 130 described herein for anatomic imaging derived 4D hemodynamics with deep learning to a current paradigm 110 in vascular imaging and hemodynamic assessment for a chosen example case of thoracic aorta assessment with 4D flow MRI 120, according to one or more examples. Exemplary vascular diseases the addressed may include aortic dilatation, aneurysm, aortic dissection, and more. The current paradigm may include one or more widely available clinical imaging tests, for example, CTA for risk assessment and treatment planning. The limitations of the current paradigm may include that the assessments and treatment planning are based on anatomic data without flow information. The current paradigm may also be limited due to poor risk stratification.

The new technology 130 described herein may introduce 4D flow training data 125 from a 4D flow MRI process 120 into a deep learning artificial intelligence system for deriving 4D hemodynamics. The 4D flow training data 125 for deep learning with the new technology 130 may be generated via the 4D flow MRI process 120 including image acquisition 121, 3D image segmentation 122. and image quantification 123. Availability of full 4D hemodynamics may be limited to select institutions. require specialized expertise, and may be associated with long scan times. This limited availability may be a rate-limiting step in clinical adoption of the future paradigm 140 for aortic hemodynamic risk-stratification and treatment planning.

In most cases, vessel anatomy and morphologic features (e.g., aorta diameter), are suboptimal risk-stratification metrics, and hemodynamic quantification based on time-resolved 3D blood flow velocities in the affected vascular region may be a complementary alternative. While aortic dimensions may be a straightforward metric for following disease progression, for example, aortic diameter, vessel diameter, or growth over time likely reflect the complex interplay of numerous factors contributing to risk in aortic and vascular diseases (e.g., vessel wall abnormalities and hemodynamics). As a result, there may be uncertainty about actual patient-specific risk which complicates decisions on patient-selection and timing of surgical intervention. In patients with bicuspid aortic valve (BAV), for example, there has been extensive debate as to the relative contribution of aortic valve stenosis and associated changes in 3D blood flow velocities and hemodynamic parameters leading to ascending aorta dilation (e.g., hemodynamic driven, valve-related aortopathy) versus underlying connective tissue abnormalities resulting in aneurysm. The potential role of hemodynamics in driving growth and associated surgical interventions or adverse outcomes has also been demonstrated in patients with many other vascular diseases, including but not limited to non-BAV-related aneurysm, intracranial aneurysm, abdominal aortic aneurysm, aortic coarctation, and aortic dissection. Some investigators have explored these questions by modeling these diseases using computational fluid dynamics (CFD). However, this method is limited by the difficulty in modeling estimates of boundary conditions and tissue mechanics of the aorta wall. Certain parameters such as ascending aorta peak velocity may be evaluated with echocardiography, although technical and acquisition limitations may prevent adequate arch or descending aorta evaluation.

Several methods exist for measuring in vivo vascular hemodynamics. Some examples include doppler ultrasound, phase contrast magnetic resonance imaging (MRI), or patient specific computational fluid dynamics. A highly specialized MRI technique called 4D flow MRI is a time-resolved, three-dimensional phase contrast technique that may facilitate direct in vivo measurement of 3D blood dynamics with full coverage of the vascular region of interest to provide comprehensive 3D blood flow visualization and 4D hemodynamic quantification. 4D flow MRI is the current standard for in vivo aorta hemodynamic quantification. 4D flow MRI has been especially well-developed in aortic imaging as the vessel's relatively large size lends itself to relatively easy acquisitions and high image quality. 4D flow MRI has been explored in many clinical translational imaging studies involving all types of aortic pathologies over the past 10+ years.

However, these techniques may require access to additional and advanced imaging, dedicated data analysis tools and software, and expertise in quantification approaches. These barriers may limit the availability of these hemodynamic metrics to specialized academic health care centers, and consequently, hemodynamic evaluation may not be performed in many patients.

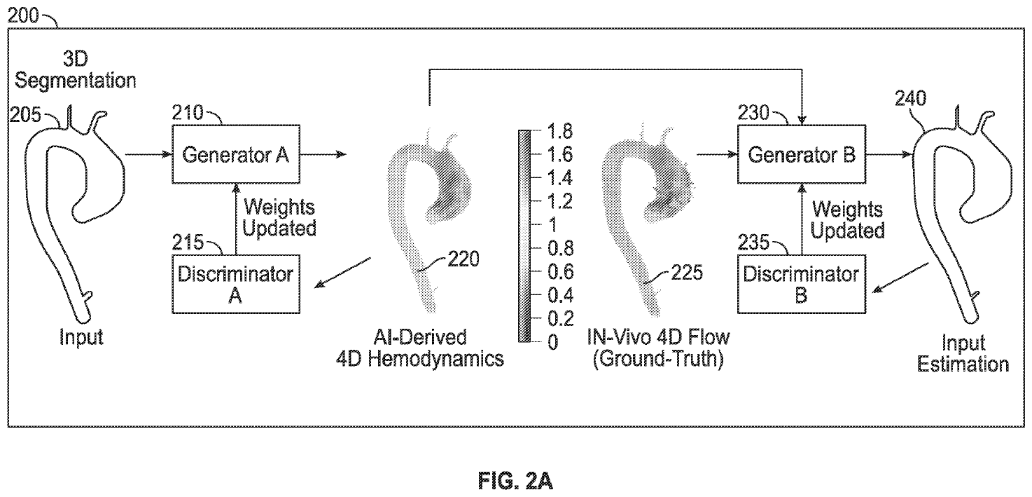

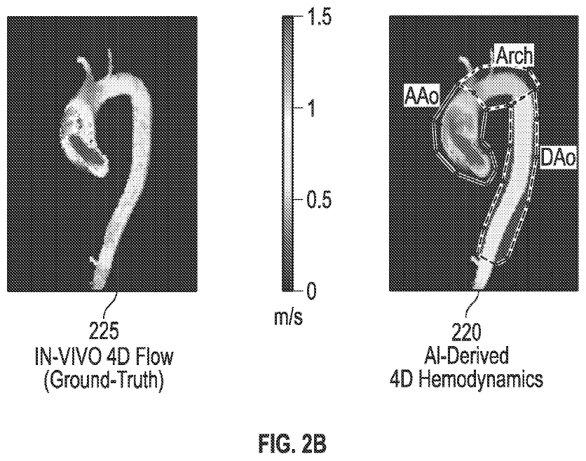

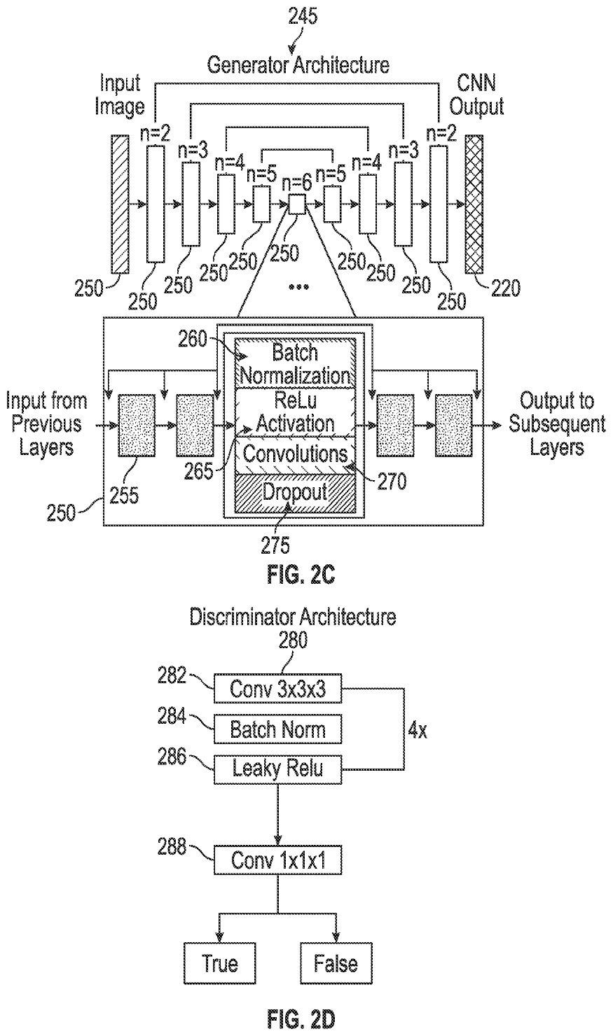

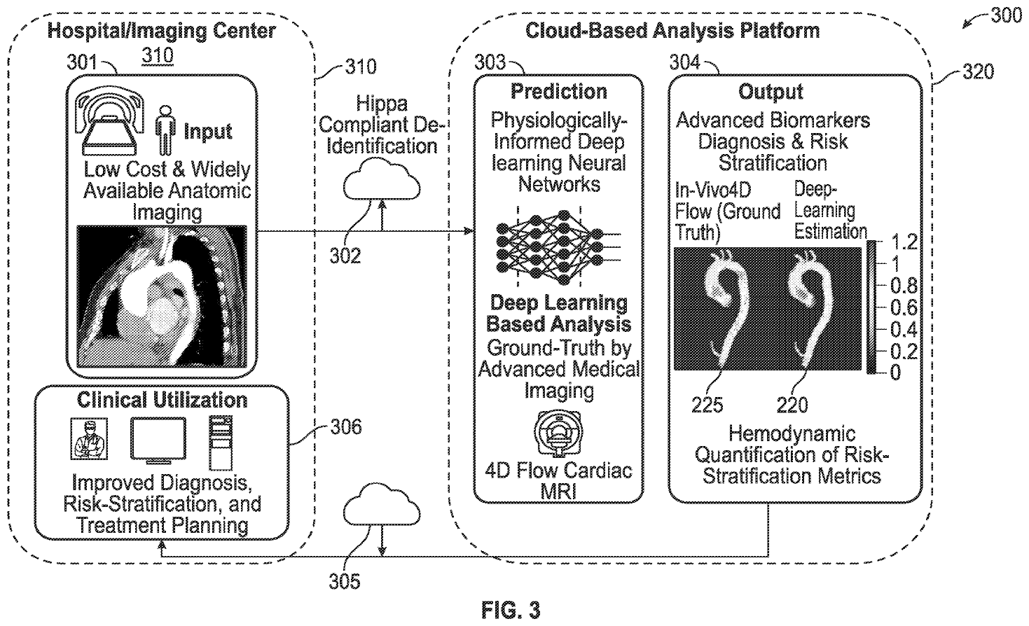

To address this lack of hemodynamic data availability in most patients, the subject technology (e.g., new technology 130) described herein may utilize imaging data obtained from standard and widely available anatomic imaging data sets (e.g., CTA or MRA of the thoracic aorta) as input data to a deep learning network. Input data may include either the full imaging data set (e.g., CTA data as acquired) or a pre-processed subset of data (e.g., 3D vessel segmentation derived from CTA data). The subject technology may then use a set of deep learning networks trained on in vivo 4D hemodynamics (e.g., 4D flow training data 125) derived from 4D flow MRI (e.g., 4D flow MRI 120) to generate spatially and temporally resolved flow velocity data that may be used (e.g., in future paradigm 140) to visualize blood flow and quantify voxel-wise hemodynamic parameters (for example, peak velocity (PV, cm/s)), forward and reverse flow (FF and RF, ml/cycle), wall shear stress (WSS, Pa), kinetic energy (KE, J), and flow stasis (e.g., as illustrated in FIG. 2 with the aorta as the vessel of interest). Data may also be passed through additional computational algorithms and/or deep learning networks for pre- or post-processing images and generating hemodynamic maps or images. quantification, and clinical reports as desired.

FIG. 2A illustrates an outline of an exemplary deep learning framework based on a CycleGAN neural network 200 that may be employed to derive 4D hemodynamics 220 from anatomic 3D segmentations 205 of an aorta. The CycleGAN neural network 200 may facilitate creation of a highly accurate 3D thoracic aorta representation and derivation of hemodynamics from CTA thoracic aorta data sets. Note that other network architectures or different anatomic input data to the deep learning network may also or alternatively be employed. The CycleGAN 200 may include a generator A 210 coupled with a discriminator A 215, and a generator B 230 coupled with a discriminator B 235. In this example, the 3D aortic geometry is provided as input data 3D segmentation 205 to the network Generator A 210. The Generator A 210 may attempt to derive 4D hemodynamics 220 while the Discriminator A 215 attempts to distinguish between the AI-derived 4D hemodynamics 220 and in-vivo 4D flow 225 (ground truth data for training and testing). The 4D hemodynamics data 220 may then be used as an input into Generator B 230 in order to derive an original input estimation 240, while Discriminator B 235 may attempt to distinguish between the original and AI-estimated geometries. Hemodynamic maps of peak velocities of the in-vivo 4D flow 225 and AI-derived systolic velocities (4D hemodynamics 220) are shown.

FIG. 2B illustrates exemplary hemodynamic maps of peak velocities of the 4D flow (in-vivo 4D flow ground-truth MRI 225) and AI-derived systolic velocities (AI-derived 4D hemodynamics 220). These AI-derived 4D hemodynamics 220 include AAo (ascending aorta). Arch. and DAo (descending aorta).

FIG. 2C illustrates an exemplary Generator convolutional neural network (CNN) architecture 245. An input image (e.g., 3D segmentation 205) may be input into a sequence of neural network nodes 250, each node 250 having an order n. The last neural network node 250 may output a CNN output (e.g., AI-derived 4D hemodynamics 220). Each neural network node 250 may include interconnected processing elements (PEs) 255, where each PE 255 may include a batch normalization module 260, a Rectified Linear Unit (reLU) activation module 265, a convolution module 270, and a dropout module 275.

FIG. 2D illustrates an exemplary Discriminator convolutional neural network (CNN) architecture 280. The Discriminator architecture 280 may include a 3×3×3 convolution module 282, a batch normalizer module 284, a leaky ReLU module 286, and a 1×1×1 convolution module 288. Outputs from the 1×1×1 convolution module 288 may be “True” when the images being compared by the discriminator architecture 280 are sufficiently similar; otherwise, outputs from the 1×1×1 convolution module 288 may be “False.”

The subject technology of FIGS. 2A-2D may be implemented in a deep-learning based segmentation tool that may provide rapid and accurate thoracic aorta segmentation from the 4D flow data set obtain via in vivo imaging. The technology may also include a tool which, based on the segmented aorta data, generates voxel-wise quantification of peak velocity (PV), forward and reverse flow (FF and RF), wall shear stress (WSS), kinetic energy (KE), and flow stasis. For WSS, patient studies using the subject technology have led to the creation of an aorta atlas of age-matched normal subject which may provide information on a percentage area of the aorta with elevated WSS relative to the age-matched controls for a given subject.

4D flow is now being used for hemodynamic-derived risk-stratification and treatment planning. The subject technology described herein is also useful for testing hypotheses to better understand how aorta hemodynamics drive the pathophysiology of aortic diseases and how these parameters may be used for risk-stratification and treatment planning.

Hemodynamic-derived risk-stratification and treatment planning is becoming increasingly validated and utilized in clinical practice. In BAV, for example, regional elevation of ascending aorta WSS quantified from 4D flow was associated with decreasing elastin in explanted aortic walls in these regions and can provide targeted aorta intervention. This finding strongly supports the role of abnormal hemodynamics driving BAV-associated ascending aorta aneurysm. It has been recently shown that higher percentage of area of elevated WSS at baseline in BAV patients is associated with more rapid aortic growth rate over at least 5 years. As result of these (and many other) findings, there is increasing demand from many radiologists, surgeons, and other clinicians to acquire this information as a part of regular follow-up imaging and treatment planning for patients with aortic disease.

Limited-availability and expertise have led to lagging clinical translation of 4D cardiovascular hemodynamics (i.e., 4D=3D+time over the cardiac cycle). Currently, the only techniques that can provide comprehensive in vivo 4D hemodynamic quantification are 4D flow MRI, 3D Doppler echocardiography, or patient specific computation fluid dynamics (CFD). However, these techniques have several key limitations that have significantly hindered widespread clinical adoption, and, therefore has limited broad ability to apply such results in most patients. Such limitations include: 1) lack of availability and local expertise for the reliable and successful completion of 4D flow MRI exams, 3D Doppler echocardiography, or patient specific computation fluid dynamics at most (especially non-academic) centers, 2) long 4D flow MRI scan times of 10-15 minutes, 3) need for dedicated and often not widely available software for hemodynamic analysts, and 4) surgeons lacking experience in planning interventions based on vascular imaging makes them less likely to order advanced hemodynamic tests.

The subject technology described herein provides a widely available. 4D hemodynamic quantification derived from standard anatomic imaging that may be used for vascular disease risk stratification and treatment planning. The subject technology may be a transformative and disruptive innovation in aorta evaluation. By providing hemodynamic quantification from standard imaging (for example, from widely available CTA instead of 4D flow), widespread clinical adoption of these tools is possible, which has never been possible before. Specific technical and clinical novelties of the subject technology are described in greater detail below.

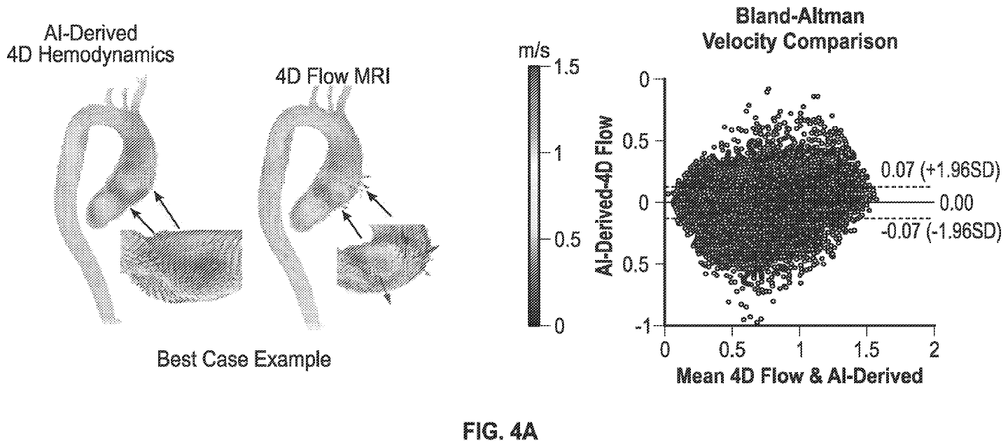

At a high-level summary, the subject technology may perform at least three primary functions. First, standard anatomic imaging data may be passed to a network for hemodynamic characterization of vessels of interest with optional preprocessing of images. Second, the network, specifically designed to learn the prediction of spatially and temporally resolved 3D blood flow velocities from the input data, may predict blood flow velocities using input data from the first primary function. Third, the subject technology may generate output data for visualization and quantification. An illustration of one potential deep learning approach is provided in FIG. 2 (described above) while a conceptual workflow for the end-user utilizing a cloud-based processing framework is outlined in FIG. 3 (described below) for a chosen example case of thoracic aorta assessment. Note that other workflows including local (e.g., non-cloud based) analysis may be performed. The subject technology may also be applied to other vascular regions in the human body.

FIG. 3 illustrates an exemplary workflow 300 for implementation of the subject technology for a chosen example case of thoracic aorta assessment. Note that other workflows including local (e.g., non-cloud based) analysis may be performed. The subject technology may also or alternatively be applied to other vascular regions in the human body. In an operation 301 at a hospital/imaging center 310, standard anatomic imaging (e.g., low cost and widely available anatomic imaging) may be acquired using standard-of-care techniques. In an operation 302, imaging data may undergo HIPPA-compliant de-identification and then be uploaded to a cloud-based analysis platform 320. In an operation 303, images may be fed into a physiologically-informed deep learning-based analysis pipeline for prediction of 4D hemodynamics. The deep learning-based analysis pipeline may include a neural network, for example, a convolutional neural network. The deep learning-based analysis may utilize ground-truth obtained by advanced medical imaging, for example, 4D flow cardiac MRI. In an operation 304, advanced hemodynamic quantification of risk-stratification metrics may be performed, and case-appropriate imaging and quantitative data may be generated. Operation 304 may output advanced biomarkers diagnosis and risk stratification. In an operation 305, outputs from operation 304 may be transferred back to the hospital/imaging center 310 (e.g., the site where images were acquired and are being clinically utilized). In an operation 306, clinicians may utilize the data obtained from the cloud-based analysis platform 320 for diagnosis, risk-stratification, and treatment planning.

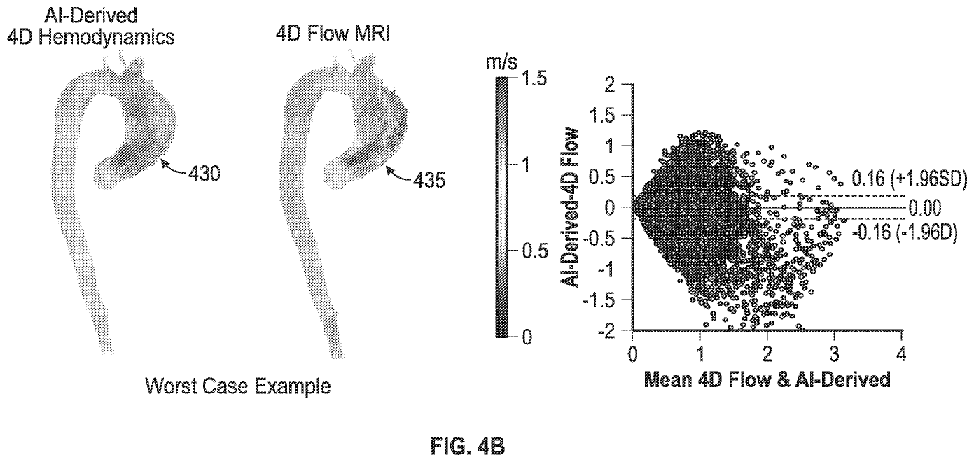

In certain aspects, for example, deep learning networks are trained and validated for performing all or part of each operation described with reference to FIG. 3. Training data for these neural networks may be derived, for example, from either expert segmentation, analysis, and interpretation of imaging data (e.g., first function), or in vivo 4D flow MRI hemodynamic data (e.g., second function). Output features (e.g., third function) may be defined based on use-case and may include two- or three-dimensional hemodynamic data mapped onto individual voxels of the vascular imaging segmentation or viewed as time-resolved velocity data (e.g., streamlines, vectors, etc.), as illustrated in FIG. 4 (discussed below). Generated data sets may be further queried for additional hemodynamic quantification and visualization either by the subject technology or end-users. Results of proof-of-concept studies in large patient cohorts have demonstrated the feasibility of this approach to provide accurate 4D hemodynamic quantification throughout the aorta in patients with aortic disease, as illustrated in FIGS. 4-7 (described below).

FIGS. 4A and 4B illustrate exemplary results from a validation study based on a total of N=1765 patients (n=1242 bicuspid aortic valve [BAV] patients, median age: 42 years; n=523 trileaflet aortic valve [TAV] patients, median age: 45 years). All N patients underwent in vivo aortic 4D flow MRI which served as ground truth data for the training and testing of a deep learning network to predict 4D aortic hemodynamics. Examples of deep learning derived vs. 4D flow MRI based 3D systolic Velocity Vector Fields for one of the best (FIG. 4A) and worst (FIG. 4B) datasets. For the best example dataset, the AI-derived velocity vector fields showed similar flow pattern compared to 4D flow MRI, including marked vortex flow in the ascending aorta. For the worst example dataset, AI-predicted velocities included an aortic valve flow jet 430 and 435 directed toward the opposite side of the aorta compared to 4D flow (black arrows). Bland-Altman velocity comparisons are provided on the right for each of FIGS. 4A and 4B, highlighting low bias and strong agreement between AI-derived and 4D flow measured aortic velocities.

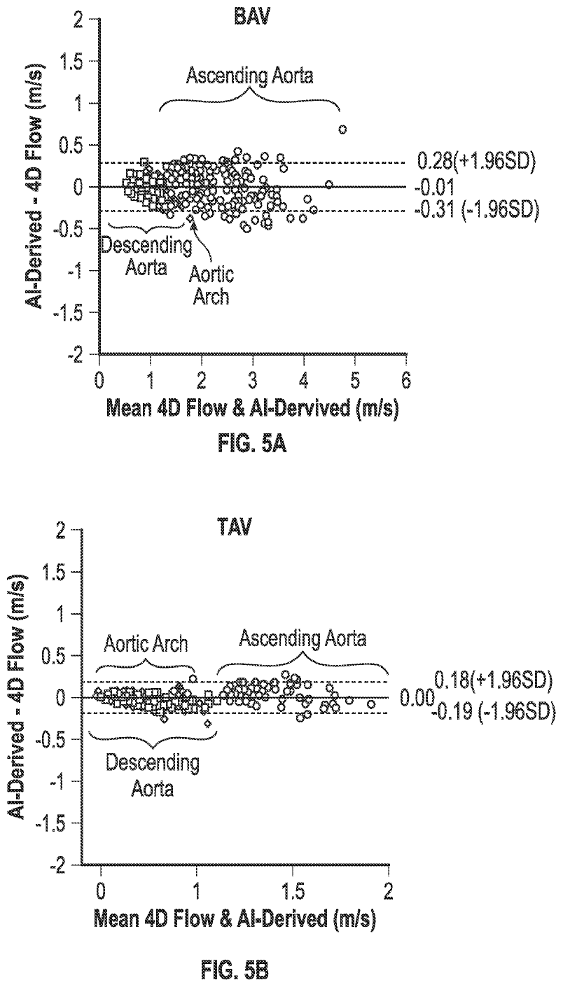

FIGS. 5A and 5B illustrate exemplary results of a validation study based on a total of N=1765 patients (n=1242 BAV patients, median age: 42 years; n=523 TAV patients, median age: 45 years). All N patients underwent in vivo aortic 4D flow MRI which served as ground truth data for the training and testing of a deep learning network to predict 4D aortic hemodynamics. Bland-Altman plots of the regional peak velocities for BAV (FIG. 5A) and TAV (FIG. 5B) patient cohorts show excellent agreement between deep learning derived ground truth data (e.g., low bias and low limits of agreement across all regions and datasets). The results include data points for ascending aorta (having higher mean values), descending aorta (having lower mean values), and aortic arch (dotted near the right outskirts of the descending aorta data points for BAV and dotted roughly throughout the descending aorta data points for TAV). Note that the data points of different types are not completely separated from each other. For example, some ascending aorta data points appear to the left of most ascending aorta data points, above some of the descending aorta data points for TAV; also, some descending aorta data points appear to the right of most descending aorta data points, at a bottom portion of a large grouping of ascending aorta data points.

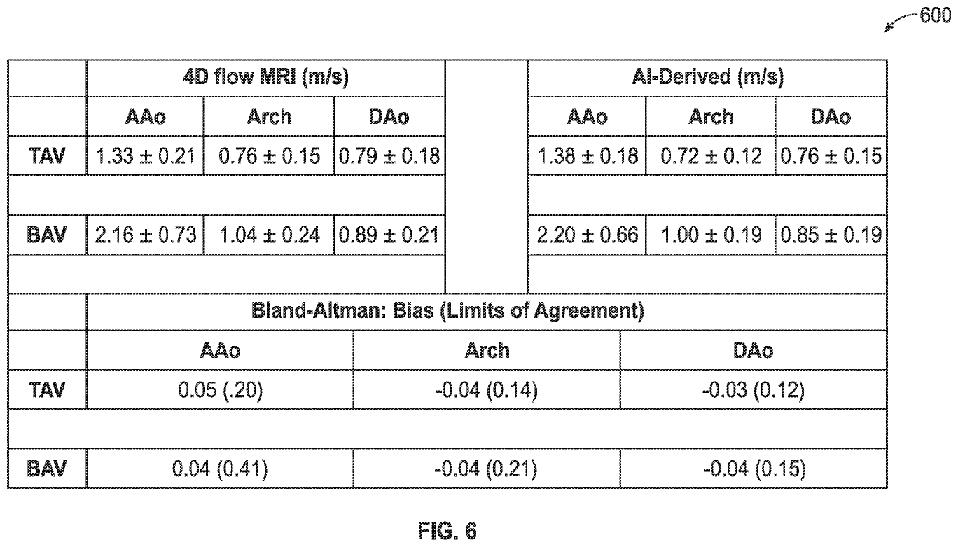

FIG. 6 shows a table 600 that illustrates exemplary results from a validation study based on a total of N=1765 patients (n=1242 BAV patients, median age: 42 years; n=523 TAV patients, median age: 45 years). All N patients underwent in vivo aortic 4D flow MRI which served as ground truth data for the training and testing of a deep learning network to predict 4D aortic hemodynamics. The table 600 summarizes peak velocities and Bland-Altman analysis results for the BAV and TAV datasets for the ascending aorta (AAo), aortic arch, and descending aorta (DAo). The average±standard deviation of the peak velocity are provided for the 4D flow MRI (left) and deep learning derived 4D hemodynamics (right).

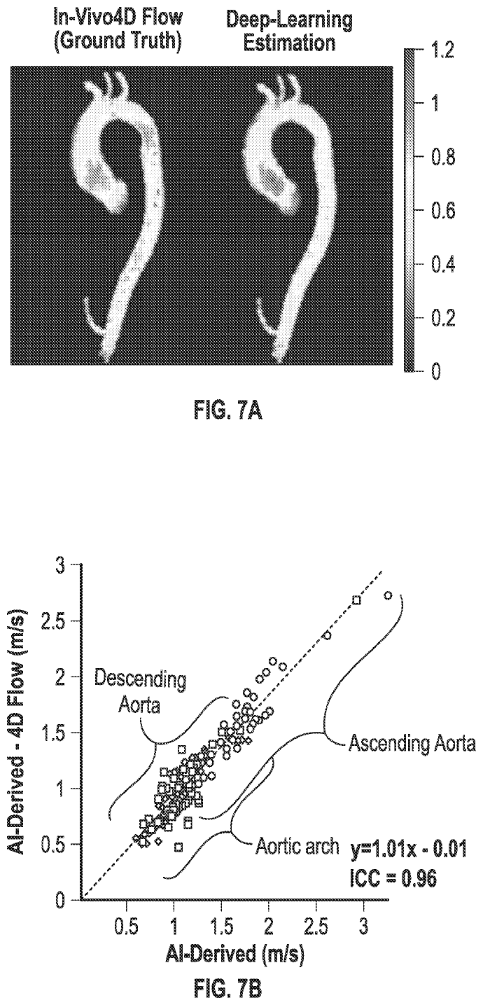

FIGS. 7A and 7B illustrate results from a validation study based on a total of N=720 patients (477 BAV patients, median age: 42 years; 243 TAV patients, median age: 45 years). All N patients underwent in vivo aortic 4D flow MRI which served as ground truth data for the training and testing of a deep learning network to predict 4D aortic hemodynamics. FIG. 7A shows an example of the deep learning hemodynamic estimations for a TAV dataset compared with an in vivo 4D flow (ground truth) dataset, which show similar systolic velocity patterns. FIG. 7B shows a plot illustrating that across all 52 testing datasets (34 BAV patients, 18 TAV patients), deep learning successfully estimated systolic aortic 3D velocities with strong agreement compared to 4D flow MRI (ground truth data) in regional peak velocity analysis across all regions (AAo, arch, DAo, ICC=0.98-0.99 [0.96-0.98-0.98-0.99].

In certain aspects, for example, the workflow (e.g., workflow 300) may facilitate users selecting an imaging dataset and vessel of interest for hemodynamic characterization, with output type and destination provided by the subject technology or defined by the user. For example, viewing environments for output data may include, but may not be limited to. standard image viewers, picture archiving and communication systems (PACS), and/or graphical user interfaces that allow voxel-wise and/or quantitative interrogation, cine review, and/or multiplanar reformation.

### Standard Vascular Imaging In Vivo Hemodynamics

Given the wide-availability, lower technical demands, and general comfort level that both radiologists and ordering physicians may have with performing and interpreting standard imaging (for example CTA, MRA, ultrasound, etc.), 4D hemodynamic quantification directly derived from standard imaging with the technology of the present disclosure may effectively address the limitations associated with other advanced hemodynamic evaluation tools (for example, 4D flow MRI, 3D Doppler echocardiography, and/or patient specific computation fluid dynamics). The subject technology may leverage 4D flow MRI, standard anatomic imaging, and AI concepts such as deep learning networks to create a cutting-edge tool which may quantify 4D hemodynamics directly from standard imaging studies. This tool may be applied on any anatomic imaging dataset (e.g., historic imaging data exported from image archives or newly acquired imaging data), may require no additional imaging, and may require little to no user interaction for advanced post-processing and/or data analysis.

### Imaging Data Acquisition, Transfer, and Preparation

As illustrated in FIG. 3, the subject technology may receive standard anatomic imaging (e.g., CTA or MRA) that has been de-identified to protect patient privacy (see operation 302). The subject technology data analysis may be performed at a specific location on a local network or transferred to a cloud-based analysis framework (e.g., the cloud-based analysis platform 320). To prepare imaging data as input for the deep learning network, the subject technology may perform some degree of pre-processing (for example, 3D segmentation of the vessel of interest) as needed. A number of deep learning techniques or combinations may be utilized for data preparation (examples include but are not limited to U-Net, V-Net, Attention U-Net, capsule based, transformer based, diffusion, etc.).

### Deep Learning of 4D Hemodynamics

The subject technology may predict 4D hemodynamics from standard anatomic imaging. To perform this function, a deep learning network (or set of networks) may be trained and validated using 4D flow MRI as ground truth with standard anatomic imaging data as input. Numerous deep learning approaches may be deployed to perform this process (e.g., CycleGAN, diffusion models, etc.). These techniques may result in a highly-accurate image-to-image translation that does not rely on large, paired datasets. The result may include a data set of spatially and temporally resolved 3D blood flow velocities that may be visualized and further interrogated for advanced 4D hemodynamic quantification. The subject technology may provide further analysis and may also make the dataset available to the end-user for additional analysis.

### Outcome: a Disruptive Tool for Aortic Disease Risk-Stratification and Treatment Planning

The subject technology may provide 4D hemodynamics without any additional dedicated hemodynamic imaging. Due to the similarities of standard imaging approaches (e.g., CTA) across imaging equipment vendors and healthcare sites, the tool of the subject technology may function with minimal or no retraining at most sites. Importantly, the tool may dramatically expand opportunities for applying 4D hemodynamic quantification to new cohorts and vascular territories. Finally, the tool may rapidly improve understanding of the role of 4D hemodynamics in risk-stratification and treatment planning by opening access to much larger patient cohorts for both retrospective and prospective research analyses.

The functions, acts or tasks illustrated in the Figures or described may be executed in a digital and/or analog domain and in response to one or more sets of logic or instructions stored in or on non-transitory computer readable medium or media or memory. The functions, acts or tasks are independent of the particular type of instructions set, storage media, processor or processing strategy and may be performed by software, hardware, integrated circuits, firmware, microcode and the like, operating alone or in combination. The memory may comprise a single device or multiple devices that may be disposed on one or more dedicated memory devices or disposed on a processor or other similar device. When functions, steps, etc. are said to be “responsive to” or occur “in response to” another function or step, etc., the functions or steps necessarily occur as a result of another function or step, etc. It is not sufficient that a function or act merely follow or occur subsequent to another. The term “substantially” or “about” encompasses a range that is largely (anywhere a range within or a discrete number within a range of ninety-five percent and one-hundred and five percent), but not necessarily wholly, that which is specified. It encompasses all but an insignificant amount.

Other systems, methods, features and advantages will be, or will become, apparent to one with skill in the art upon examination of the figures and detailed description. It is intended that all such additional systems, methods, features and advantages be included within this description, be within the scope of the disclosure, and be protected by the following claims.

What is claimed is:

1. A computer-implemented method for non-invasive assessment of vascular 4D hemodynamics, the method comprising:
receiving standard anatomic imaging data at a local network or cloud-based analysis platform;
identifying a vessel of interest from the received anatomic imaging data;
deriving hemodynamic features from the vessel of interest from the received anatomic imaging data using deep learning by inputting the received anatomic imaging data into a deep learning network; and
calculating 4D hemodynamic parameters and generating output data based on the hemodynamic features derived from the vessel of interest.

2. The method of claim 1, wherein identifying the vessel of interest comprises pre-processing the anatomic imaging data that is received.

3. The method of claim 1, wherein identifying the vessel of interest comprises performing 3D segmentation of the vessel of interest.

4. The method of claim 1, further comprising passing the received anatomic images to a deep learning network for performing 4D hemodynamic quantification and pre-processing of anatomic imaging data of the vessel of interest on the deep learning network.

5. The method of claim 4, further comprising training the deep learning network using expert labeled datasets of previously obtained vascular imaging data.

6. The method of claim 1, further comprising deriving spatially and temporally resolved 3D blood flow velocities in the vessel of interest from the received anatomic imaging data using deep learning by inputting the received anatomic imaging data into a deep learning network.

7. The method of claim 1, wherein calculating 4D hemodynamic parameters is performed on a deep learning network from anatomic imaging data.

8. The method of claim 7, wherein the deep learning network is trained using expert-analyzed 4D flow MRI data as ground truth data.

9. The method of claim 1, further comprising displaying the output data that is generated on a device selected from the group consisting of an image viewer, a picture archiving and communication system, and a graphical user interface.

10. The method of claim 9, wherein the graphical user interface is configured to facilitate at least one of quantitative interrogation, cine review, and multiplanar reformation.

11. A system for for non-invasive assessment of vascular 4D hemodynamics comprising:
at least one device including a hardware computing processor;
the system being configured to perform operations comprising:
receiving standard anatomic imaging data at a local network or cloud-based analysis platform;
identifying a vessel of interest from the received anatomic imaging data;
deriving hemodynamic features from the vessel of interest from the received anatomic imaging data using deep learning by inputting the received anatomic imaging data into a deep learning network; and
calculating 4D hemodynamic parameters and generating output data based on the hemodynamic features derived from the vessel of interest.

12. The system of claim 11, wherein identifying the vessel of interest comprises pre-processing the anatomic imaging data that is received.

13. The system of claim 11, wherein identifying the vessel of interest comprises performing 3D segmentation of the vessel of interest.

14. The system of claim 11, wherein the operations further comprise passing the received anatomic images to a deep learning network for performing 4D hemodynamic quantification and pre-processing of anatomic imaging data of the vessel of interest on the deep learning network.

15. The system of claim 14, wherein the operations further comprise training the deep learning network using expert labeled datasets of previously obtained vascular imaging data.

16. The system of claim 11, wherein the operations further comprise deriving spatially and temporally resolved 3D blood flow velocities in the vessel of interest from the received anatomic imaging data using deep learning by inputting the received anatomic imaging data into a deep learning network.

17. The system of claim 11, wherein calculating 4D hemodynamic parameters is performed on a deep learning network from vascular imaging data.

18. The system of claim 17, wherein the deep learning network is trained using expert-analyzed 4D flow MRI data as ground truth data.

19. The system of claim 1, wherein the operations further comprise displaying the output data that is generated on a device selected from the group consisting of an image viewer, a picture archiving and communication system, and a graphical user interface.

20. The system of claim 19, wherein the graphical user interface is configured to facilitate at least one of quantitative interrogation, cine review, and multiplanar reformation.
